(19) United States
(12) Patent Application Publication
(10) Pub. No.: US 2022/0324831 A1
(43) Pub. Date: Oct. 13, 2022

(54) STABLE CRYSTALLINE APALUTAMIDE IN PURE FORM, AND PROCESS FOR THE PREPARATION THEREOF

(71) Applicant: OLON S.P.A., Rodano (MI) (IT)

(72) Inventors: Valentina Grande, Rodano (MI) (IT); Alessia Manfredi, Rodano (MI) (IT); Jacopo Bonanomi, Rodano (MI) (IT); Gabriele Ferretti, Rodano (MI) (IT); Barbara Novo, Rodano (MI) (IT); Mara Sada, Rodano (MI) (IT); Giorgio Bertolini, Rodano (MI) (IT)

(73) Assignee: OLON S.P.A., Rodano (MI) (IT)

(21) Appl. No.: 17/753,645

(22) PCT Filed: Sep. 7, 2020
(86) PCT No.: PCT/EP2020/074975

§ 371 Date: Mar. 10, 2022

(30) Foreign Application Priority Data
Sep. 10, 2019 (IT) 102019000015974

(51) Int. Cl. C07D 401/04

(52) U.S. Cl. CPC C07D 401/04; C07B 2200/13

(57) ABSTRACT

The present invention relates to a novel non-solvated crystalline form of apalutamide in pure, stable form, and the process for the preparation thereof.

The present invention relates to a novel stable non-solvated crystalline form of apalutamide, and the process for the preparation thereof.

### PRIOR ART

Apalutamide, 4-{7-[6-cyano-5-(trifluoromethyl)pyridin-3-yl]-8-oxo-6-sulphanylidene-5,7-diazaspiro[3.4]octan-5-yl}-2-fluoro-N-methylbenzamide, disclosed in U.S. Pat. No. 8,445,507, is currently used to treat non-metastatic castration-resistant prostate cancer.

Several crystalline forms and an amorphous form of apalutamide are known: WO2013184681 discloses crystalline forms A, B, C, D, E, F, G, I and J, and their use for the preparation of capsules. The most stable form of those disclosed in WO2013184681 is Form B.

The other crystalline forms are solvated or poorly stable, and can form isostructural solvates, depending on the temperature and humidity conditions to which they are exposed, and on the various solvents used for the crystallisation. In particular:
form A may be non-solvated, solvated or hydrated;
form C obtained from isopropanol, anisole or mixtures of isopropanol and water is a solvate;
form D obtained from methyl tert-butyl ether is a solvate;
form E obtained from dimethylsulphoxide is a 1:1 solvate;
form G obtained from 2-methoxyethanol is a 1:1 solvate;
form J obtained from acetone:water is a solvate.

If the crystallisation solvent remains trapped in the crystalline cell it can only be removed by melting the product. Conversely, if the solvent is not retained by the crystal, it causes the collapse of the crystalline cell, leading to the formation of a mixture of the amorphous form and metastable crystalline forms. Both cases give rise to problems in formulating stable pharmaceutical compositions.

### DESCRIPTION OF THE INVENTION

It has now been found that a crystalline solvated form of apalutamide which contains a lower level of impurities than form B can be obtained by crystallising crude apalutamide in acetonitrile or in a mixture of acetonitrile and another solvent.

The form obtained is a solvate of acetonitrile having an oxo-apalutamide content of formula (II)

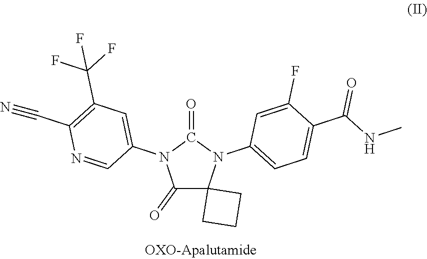 [SMILES: CNC(=O)c1ccc(N2C(=O)N(c3cnc(C#N)c(C(F)(F)F)c3)C(=O)C23CCC3)cc1F]

lower than that of form B, for which purity values of about 99% are reported in the literature.

It has also been found that the solvated form of acetonitrile can be suitably dried to give a stable form, called form Y, with an acetonitrile content lower than 410 ppm (ICH Guideline limit), having a high degree of purity (>99.8%), and characterised by an oxo-apalutamide content of less than 0.05%. The resulting form Y is particularly suitable for the preparation of pharmaceutical formulations.

### DESCRIPTION OF FIGURES

FIG. 1: Shows the XRPD spectrum of crystalline form Y (Cu K-Alpha 1-2-Theta λ=1.54060);

FIG. 2: Shows the IR spectrum of form Y;

FIG. 3: Shows the DSC of form Y, with a thermal gradient ranging from 40.0° C. to 220° C. at 10.0° C./minute;

FIG. 4: Shows the XRPD spectrum recorded at the Cu—K-alpha wavelength (λ=1.54060) of the acetonitrile-solvated form of apalutamide;

FIG. 5: Shows the IR spectrum of the acetonitrile-solvated form of apalutamide;

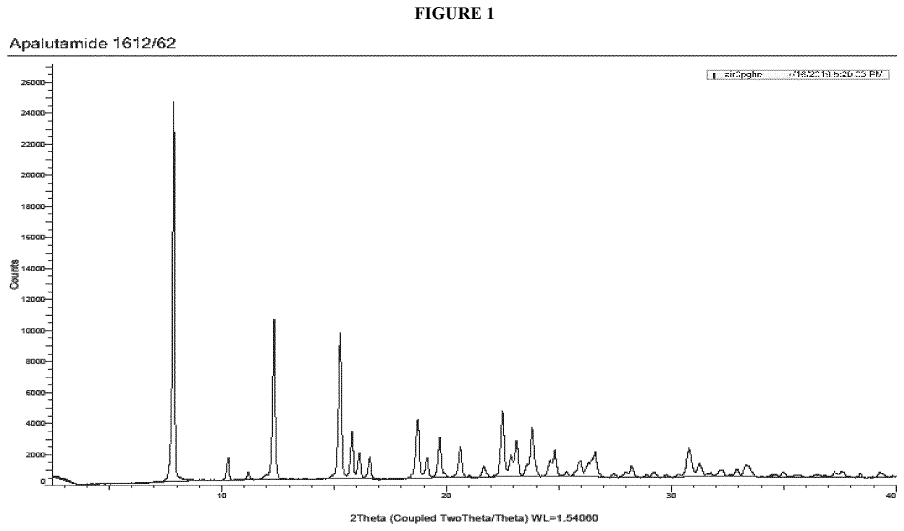

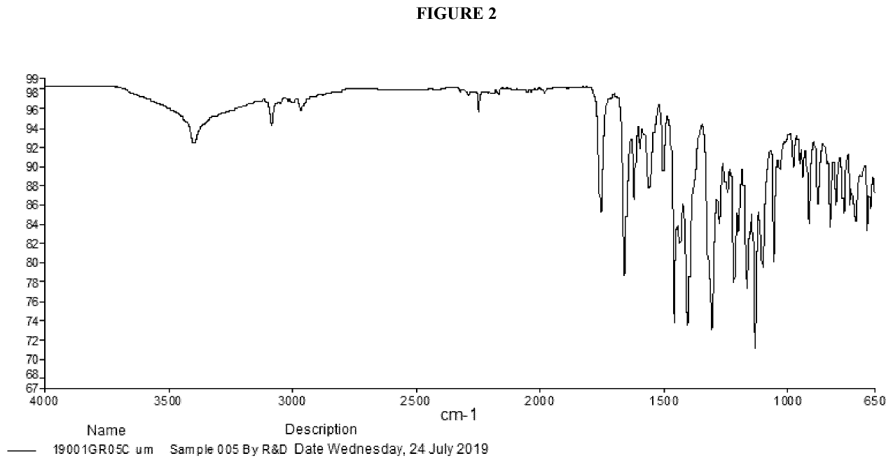

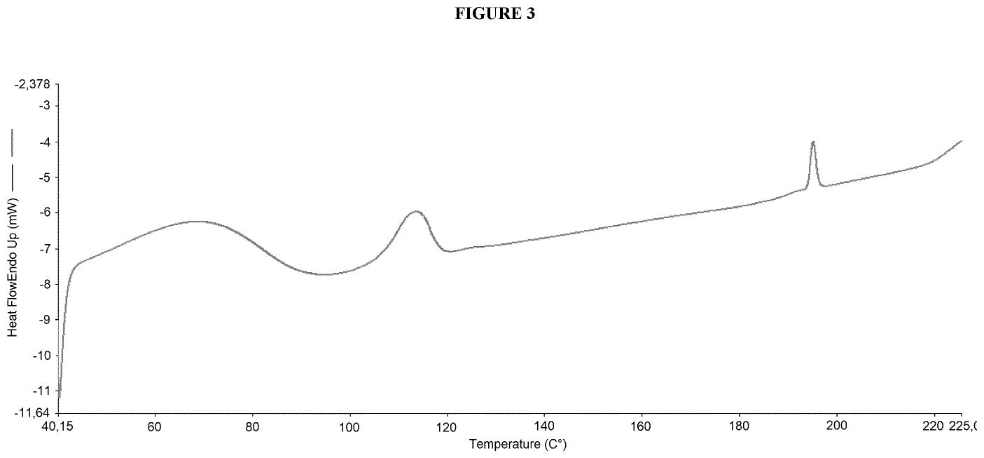

FIG. 6: Shows the DSC with thermal gradient ranging from 40.0° C. to 220° C. at 10.0° C./minute of the acetonitrile-solvated form of apalutamide. Form Y according to the invention presents the following characteristics:
i. an X-ray diffraction spectrum (XRPD) comprising peaks at 7.8°±0.2°2θ, 10.3°±0.2°2θ, 12.3°±0.2°20, 15.3°±0.2°2θ, 18.7°±0.2°2θ and 22.5°±0.2°2θ, as shown in FIG. 1;
ii. an IR spectrum as shown in FIG. 2;
iii. a DSC profile with a thermal gradient ranging from 40.0° C. to 220° C. at 10.0° C./minute, as shown in FIG. 3;
iv. an acetonitrile content of less than 410 ppm.

The invention also relates to a crystalline acetonitrile solvate of apalutamide useful as an intermediate for the preparation of form Y.

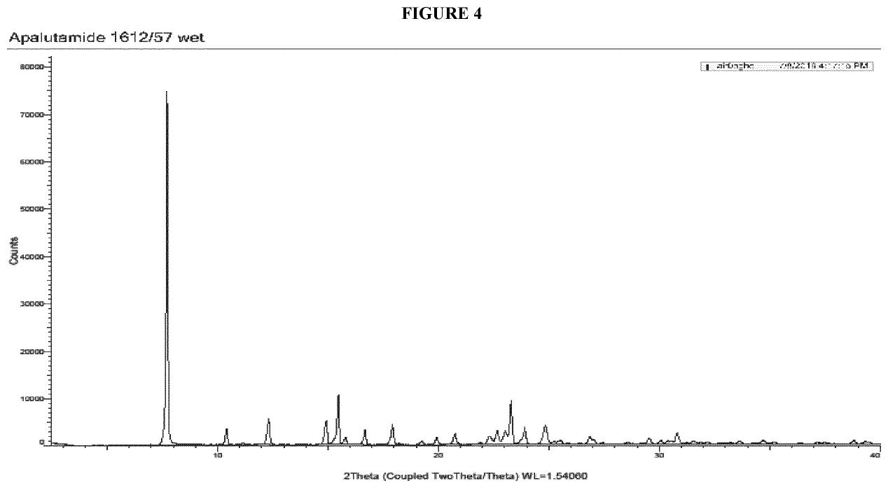

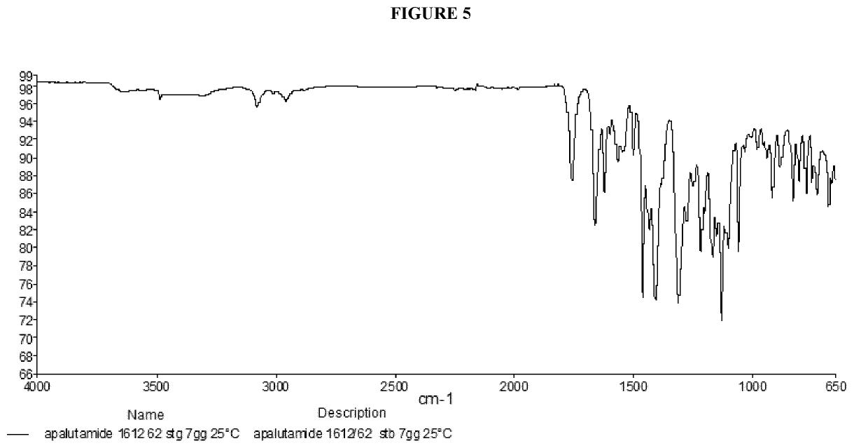

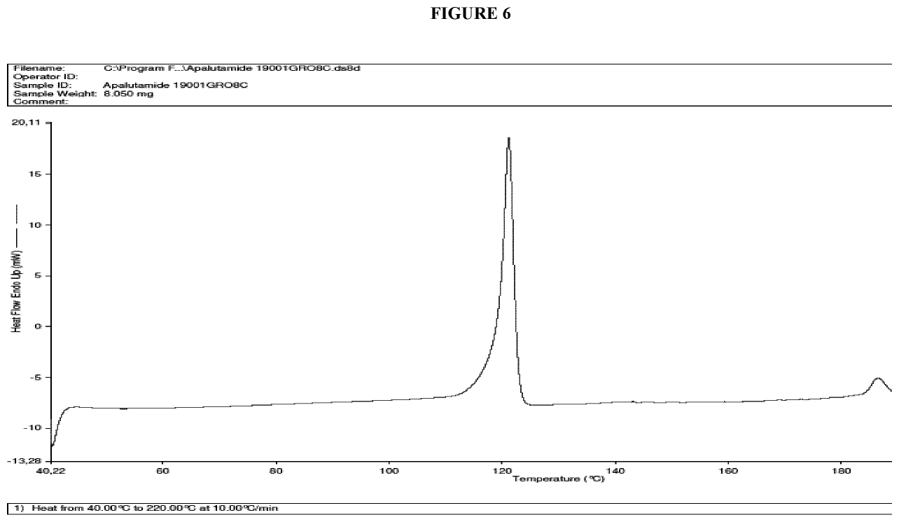

The acetonitrile-solvated form of apalutamide has the following characteristics:
i. an X-ray diffraction spectrum (XRPD) comprising peaks at 7.7°±0.2°2θ, 10.4°±0.2°2θ, 12.3°±0.2°2θ, 15.4°±0.2°2θ, 17.9°±0.2°2θ and 22.4°±0.2°2θ, as shown in FIG. 4;
iii. an IR spectrum as shown in FIG. 5;
iv. a DSC profile with a thermal gradient ranging from 40.0° C. to 220° C. at 10.0° C./minute, as shown in FIG. 6, and an oxo-apalutamide content of less than 0.05%.

A further object of the invention is the process for preparation of form Y, which comprises crystallisation of crude apalutamide from acetonitrile or from a mixture of acetonitrile and an acetonitrile-miscible solvent, followed by drying at 30-90° C. in the presence of water for a period of 3-48 h.

The acetonitrile-miscible solvent is selected from water, methanol, acetone, tetrahydrofuran, toluene, cyclohexane, dimethyl carbonate, cyclopentyl methyl ether, dimethylsulphoxide and dichloromethane.

The ratio between apalutamide and solvent ranges between 1:1 and 1:30, preferably 1:5.

The apalutamide is first suspended in the solvent, wherein it is solubilised by heating to the boiling point of the solvent or mixture of solvents used, preferably at a temperature ranging between 25 and 90° C., more preferably at 30-70° C.

Precipitation can be obtained by cooling to a temperature ranging between 0 and 25° C., preferably to a temperature ranging between 10 and 20° C., or by adding an anti-solvent selected from water, toluene and cyclopentyl methyl ether, preferably water.

The resulting product is filtered and dried to remove the acetonitrile.

The drying process is conducted under vacuum at a temperature of 30-90°, preferably 55-70°, in the presence of controlled humidity to prevent the collapse of the crystalline cell.

Drying is conducted at a humidity rate ranging between 20-50%, preferably 40%, for a period of 5-100 h, preferably 48 h.

The following examples illustrate the invention in greater detail.

### EXAMPLE 1

10.0 g of apalutamide is suspended in 50 ml of acetonitrile, and the mixture is heated to T=65° C. The resulting solution is then cooled to T=15° C., and maintained at said temperature until the product crystallises. 50 ml of water is added, and the suspension is maintained at T=15° C. for about 1 h, then filtered to obtain 9.5 g of apalutamide in acetonitrile-solvated form.

### EXAMPLE 2

10.0 g of apalutamide is suspended in 50 ml of acetonitrile, and the mixture is maintained under stirring at T=25° C. After about 30 minutes a small amount of apalutamide in acetonitrile form is added, and the suspension is maintained at T=25° C. for 72 h, then filtered to obtain 9.5 g of apalutamide in acetonitrile-solvated form.

### EXAMPLE 3

10.0 g of apalutamide is suspended in 50 ml of acetonitrile, and the mixture is heated to T=65° C. The resulting solution is then concentrated at low pressure to a small volume, and the formation of a solid is observed. The resulting suspension is filtered to obtain 9.5 g of apalutamide in acetonitrile-solvated form.

### EXAMPLE 4

10.0 g of apalutamide is suspended in 50 ml of acetonitrile, and the mixture is heated to T=65° C. until completely dissolved. The solution is cooled to 50° C., 50 ml of water is then slowly added, and the precipitation of a white solid is observed.

The resulting suspension is cooled to T=25° C. and maintained at said temperature for about 1 h, then filtered to obtain 9.5 g of apalutamide in acetonitrile-solvated form.

### EXAMPLE 5

10.0 g of acetonitrile-solvated apalutamide is placed in a stove at the temperature of 60° C. in the presence of water, and dried under vacuum under said conditions for about 48 h.

When drying is complete, 10.0 g of apalutamide form Y having purity (HPLC) >99.8% and an acetonitrile content (GC) <410 ppm is discharged.

### EXAMPLE 6: COMPARISON OF THE PURITY OF FORMS B AND Y

10 g of apalutamide (purity 99.2%, oxo-apalutamide impurity 0.2%) is suspended in 80 mL of an acetone/cyclohexane mixture (2:8) at room temperature for 2 h, then cooled at 10° C. for 1 h and filtered, obtaining 8 g of apalutamide form B (purity 99.72%, oxo-apalutamide impurity 0.16%).

10 g of apalutamide (purity 99.2%, oxo-apalutamide impurity 0.2%) is suspended in 50 ml of acetonitrile, and the mixture is heated at T=65° C. until completely dissolved. The solution is cooled to 50° C., 50 ml of water is then slowly added, and the precipitation of a white solid is observed.

The resulting suspension is cooled to T=25° C. and maintained at said temperature for about 1 h, then filtered to obtain 8.5 g of apalutamide in acetonitrile-solvated form. The resulting solid is placed in a stove at the temperature of 60° C. in the presence of water, and dried under vacuum under said conditions for about 48 h.

When drying is complete, 9.5 g of apalutamide form Y (purity 99.88%, oxo-apalutamide impurity 0.03%) is obtained.

1. Apalutamide crystalline form Y, having the following characteristics:
i. an x-ray diffraction spectrum (XRPD) as reported in FIG. 1 including peaks at 7.8°±0.2°2θ, 10.3°±0.2°2θ, 12.3°±0.2°2θ, 15.3°±0.2°2θ, 18.7°±0.2°2θ and 22.5°±0.2°2θ (Cu-K-alpha 1° λ=1.54060);
ii. an IR spectrum as reported in FIG. 2;
iii. a DSC profile with a thermal gradient ranging from 40.0° C. to 220° C. at 10.0° C./minute as reported in FIG. 3;
iv. an acetonitrile content lower than 410 ppm.

2. A process for the preparation of apalutamide form Y of claim 1 which comprises crystallizing crude apalutamide from acetonitrile or a mixture of acetonitrile and an acetonitrile-miscible solvent, followed by drying at 30-90° C. in the presence of water for a time of 3-18 h.

3. The process according to claim 2 wherein the acetonitrile-miscible solvent is selected from water, methanol, acetone, tetrahydrofuran, toluene, cyclohexane, dimethyl carbonate, cyclopentyl methyl ether, dimethylsulphoxide and dichloromethane.

4. The process according to claim 2 wherein the apalutamide-solvent ratio ranges from 1:1 to 1:30.

5. The process according to claim 2 wherein crystallisation is carried out by cooling at a temperature ranging from 0 to 25° C., or by addition of an anti-solvent selected from water, toluene and cyclopentyl methyl ether.

6. The process according to claim 2 wherein drying is carried out at 55-70° C. with humidity ranging from 20 to 50% in 5-100.

7. Apalutamide crystalline acetonitrile solvated form, which has the following characteristics:
i. an x-ray diffraction spectrum (XRPD) including peaks at 7.7°±0.2°2θ, 10.4°±0.2°2θ, 12.3°±0.2°2θ, 15.4°±0.2°2θ, 17.9°±0.2°2θ and 22.4°±0.2°2θ (Cu—K-alpha 1° λ=1.54060) as reported in FIG. 4;
ii. an IR spectrum as reported in FIG. 5;
iii. a DSC profile with a thermal gradient ranging from 40.0° C. to 220° C. at 10.0° C./minute as reported in FIG. 6;
iv. an oxo-apalutamide content of less than 0.05%.

8. The process according to claim 4, wherein the apalutamide-solvent ratio ranges from 1:5.

9. The process according to claim 5, wherein crystallization is carried out by cooling at a temperature ranging from 10 to 20° C.

10. The process according to claim 5, wherein the crystallization is carried out by addition of water.

11. The process according to claim 6, wherein drying is carried out with humidity ranging at 40%.

12. The process according to claim 6, wherein drying is carried out in 48 hours.
